(19) United States
(12) Patent Application Publication
(10) Pub. No.: US 2021/0170065 A1
(43) Pub. Date: Jun. 10, 2021

(54) Pairing of band-aid type bandage with a wet wipe pouch

(71) Applicant: RENATA IRINA DUMITRASCU, New Glarus, WI (US)

(72) Inventor: RENATA IRINA DUMITRASCU, New Glarus, WI (US)

(21) Appl. No.: 16/706,755

(22) Filed: Dec. 8, 2019

(51) Int. Cl. A61L 15/44

(52) U.S. Cl. CPC A61L 15/44; A61L 2300/404

(57) ABSTRACT

A method and system for pairing a band-aid type of bandage with a wet wipe pouch adhered to the exterior, non-wound facing side of said bandage.

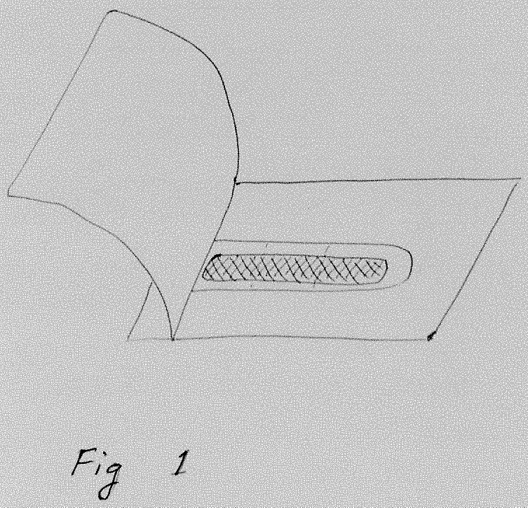

### BRIEF SUMMARY OF THE INVENTION

A method and system for pairing a band-aid type of bandage with a wet wipe pouch adhered to the exterior, non-wound facing side of said bandage. Such wet wipe can be imbibed with antiseptic or wound healing properties according to FDA rules and regulations pertaining to such products and its intent is either for cleansing on or around the wound before applying the bandage or cleansing around the wound and the adhesive left on skin by the bandage after bandage removal.

### BACKGROUND OF THE INVENTION

Minor cuts or wounds that can be covered with a commonly used band-aid type bandage usually require some kind of cleaning of the wound before application of the bandage. This is often difficult to do when one is out and about without access to soap and clean water. People may carry a band-aid but few carry a wet wipe. This invention combines the two creating a new product that addresses this need.

### DESCRIPTION OF THE INVENTION

This invention combines two existing products into a new product. The novelty lies in the two products being combined and attached to create a superior product with new capabilities.

Wet wipes with antiseptic or wound healing promotion properties can be attached directly to the non-wound facing side of the band-aid type bandage and the resulting bandage has the advantage of being a portable would treatment set that allows the user to either cleanse and treat the wound before applying the bandage or clean the wound area and surrounds, often affected by adhesive remainder, after the removal of the bandage.

The wet wipe pouch is attached to the superior part of the bandage in such a way to allow air to flow between the two objects. This can be achieved by employing partial adhesion as relating to the total surface making contact between the two surfaces.

The wet wipe pouch is peeled off the band-aid bandage, opened, the wet wipe extracted, used and discarded. The pouch that the wet wipe came in is also discarded leaving the band-aid intact.

### OTHER DESIGNS

Another version of this invention involves simply pairing the wet wipe pouch with the band-aid type bandage in the same individual, sterile package without necessarily adhering them together.

### Definitions

The term “band-aid” shall refer to the type of adhesive bandage that is in common use for covering wounds under that brand name.

The term “wet wipe” shall refer to the type of pouch containing a flexible, absorbent sheet imbibed with liquid typically used for wiping the skin after which it is discarded. For purposes of this invention the wet wipe can be imbibed with any substance commonly used for treating and cleaning wounds as approved by the FDA.

1. A new combination band-aid type of bandage and wet wipe pouch attached to the non-wound facing side of said bandage.

2. The above-mentioned bandage combination type whereby the wet wipe pouch is attached in such a way to be easily removable while leaving bandage intact.

3. The above-mentioned bandage combination type whereby the wet wipe pouch and the bandage are not attached but simply packaged in the same individual, sterile package.
